(19) United States
(12) Patent Application Publication
(10) Pub. No.: US 2007/0038088 A1
(43) Pub. Date: Feb. 15, 2007

(54) MEDICAL IMAGING USER INTERFACE AND CONTROL SCHEME

(76) Inventors: Collin A. Rich, Ypsilanti, MI (US); Clement James Goebel III, Ypsilanti, MI (US)

Correspondence Address: SCHOX PLC, 209 N. MAIN STREET #200, ANN ARBOR, MI, 48104, US

(21) Appl. No.: 11/462,693

(22) Filed: Aug. 4, 2006

Related U.S. Application Data
(60) Provisional application No. 60/705,320, filed on Aug. 4, 2005.

(51) Int. Cl. A61B 8/00

(52) U.S. Cl. 600/437

(57) ABSTRACT

An embodiment of the invention includes a method of capturing a series of images of a subject patient with a medical probe having a magnification factor, a frame rate, and an image resolution. The method includes the determining a relative motion between a subject patient and a medical probe and comparing the relative motion to a threshold. The method also includes, the method includes selecting a motion mode for the medical probe and capturing a first series of images of the subject patient with the medical probe in the motion mode if the relative motion is greater than the threshold. The method also includes selecting a stability mode for the medical probe and capturing a second series of images of the subject patient with the medical probe in the stability mode if the relative motion is less than the threshold.

### CROSS-REFERENCE TO RELATED APPLICATIONS

This application claims the benefit of U.S. Provisional Application Ser. No. 60/705,320, entitled “Ultrasound Imaging With Improved User Interface” filed 04 Aug. 2005, which is incorporated in its entirety by this reference.

### TECHNICAL FIELD

This invention relates generally to the medical imaging field, and more specifically to an improved user interface and control scheme in the medical imaging field.

### BACKGROUND

Since the 1950s, ultrasound imaging has progressed from simple, analog A-mode imaging to far more sophisticated digital B-mode and color Doppler systems, which allow users (i.e., medical specialists) to view anatomy and pathologic conditions of a subject patient. There are, however, two problems in ultrasound imaging that arise from the relative motion between the probe and the subject patient. The relative motion may be caused either by motion of the probe (e.g., intentional repositioning to change the image target and/or orientation) relative to the subject patient, or by motion of the subject patient (e.g. breathing, heartbeat, etc.) relative to an otherwise stationary probe.

The first problem arises because images of the subject patient may be greatly magnified to increase the apparent size of a small portion of the subject patient. Even relatively small movements between the probe and the subject patient may cause dramatic changes in greatly magnified images, which often leads to disorientation of the user and/or loss of the intended imaging area.

The second problem arises because the images may be captured in a high-resolution and/or 3D, which tends to require multiple ultrasound firings per frame and tends to decrease the overall frame rate of the system. Relative motion between the probe and the subject patient may cause distortion and smearing, which obscures fine detail of the subject patient, and may cause a time lag between a movement of the medical probe and an update of the captured images, which often leads to disorientation of the user.

Thus, there is a need in the medical imaging field to create an improved user interface and control scheme that reduces or eliminates at least one of these problems in conventional imaging systems. This invention provides such improved user interface and control scheme.

### BRIEF DESCRIPTION OF THE FIGURES

FIG. 1 is a flowchart of the method of the first preferred embodiment.

FIG. 2 is a schematic of the system of the second preferred embodiment.

FIG. 3 is an example of a subject patient in a 2D cross-sectional view, while FIG. 4 is an example of a subject patient in a 3D segmented view.

### DESCRIPTION OF THE PREFERRED EMBODIMENTS

The following description of the preferred embodiment of the invention is not intended to limit the invention to these preferred embodiments, but rather to enable any person skilled in the art of medical imaging to make and use this invention.

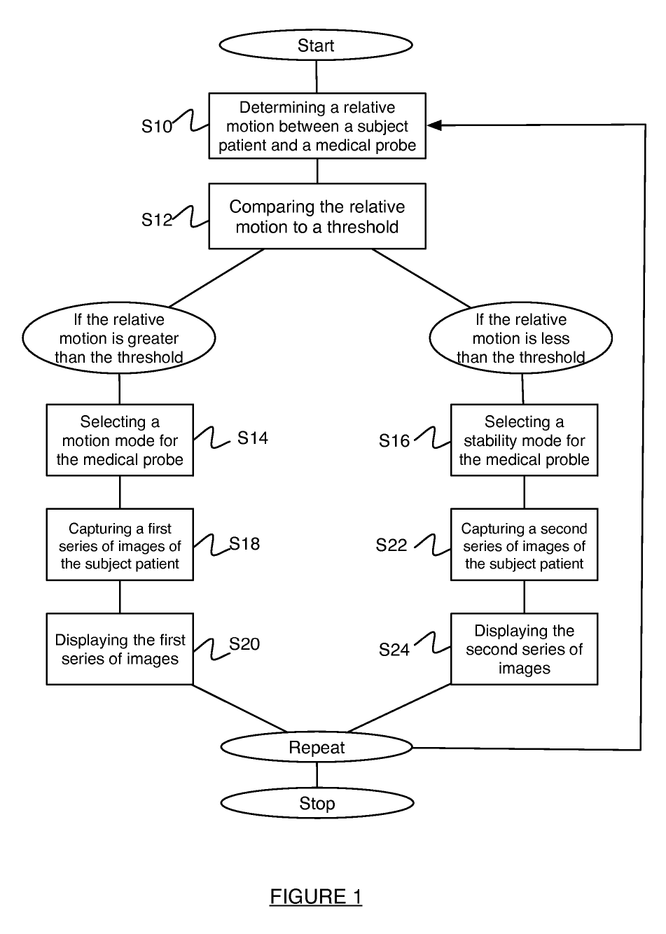

As shown in FIG. 1, an embodiment of the invention includes a method of capturing a series of images of a subject patient with a medical probe having a magnification factor, a frame rate, and an image resolution. The method includes the determining a relative motion between a subject patient and a medical probe (step S10) and comparing the relative motion to a threshold (step S12). The method also includes the selecting a motion mode for the medical probe (step S14) if the relative motion is greater than the threshold, and selecting a stability mode for the medical probe (step S18) if the relative motion is less than the threshold.

The step S10 of determining a relative motion between a subject patient and a medical probe functions to provide information for the steps of selecting the motion mode or the stability mode for the medical probe. The step preferably includes monitoring the relative motion between the subject patient and the medical probe and comparing the relative motion to a threshold. The step may, alternatively, include any suitable substep(s) to provide information on the relative motion of the medical probe and the subject patient. The relative motion between the medical probe and the subject patient may be sensed by either or both of the following ways: detection by image-processing or by physical detection.

In the image-processing variation, the step of monitoring includes capturing an initial series of images of the subject patient with the medical probe and analyzing the initial series of images. The step of analyzing the initial series of images includes motion tracking (also known as “video tracking”), frame correlation (also known as “intraframe correlation”), speckles tracking, or any other suitable image processing method or combination of image processing methods. The image-processing variation, while being more complex than the physical detection method, detects both motion of the medical probe relative to the subject patient as well as motion (internal and/or external) of the subject patient relative to the probe.

In the physical detection variation, the step of monitoring includes monitoring the motion of the medical probe relative to the environment, while assuming that the subject patient is relatively stationary. The step of monitoring preferably includes sensing acceleration forces, but may alternatively include sensing Doppler effects, sensing magnetic fields, or any other suitable method. Although this physical detection variation is simpler than the image-processing variation, it is generally effective only for user-initiated motion of the medical probe and would likely not detect motion of the subject patient relative to a stationary medical probe.

The step S12 of comparing the relative motion to a threshold functions to facilitate the selection of a proper mode for the medical probe. The threshold may be quantified as the displacement, speed, acceleration of an object captured by the medical probe, as temporal redundancy of the images captured by the medical probe, or as any other suitable quantity. The threshold may be manually adjustable by the user and may be dynamically adjustable by a processor or other suitable device of the medical probe. The threshold may be based on the entirety of the image captured by the medical probe, or a subset or portion of the image captured by the medical probe. For example, the step S12 may include comparing the relative motion of the medical probe and the rib cage of the subject patient, while ignoring the relative motion of the medical probe and the beating heart of the subject patient. The comparison may also ignore the jitters, or repeating relative motion, such as relative motion produced by a beating heart or a expanding lung.

The steps S14 and S16 of selecting a proper mode for the medical probe functions to adjust particular parameters of the medical probe and to reduce or eliminate user disorientation. The actual switching from a first mode to a second mode preferably includes a hysteresis, or intentionally time delay, to increase the user experience. The hysteresis may be preset, machine-learned, or user-set. There are several variations for the motion mode and the stability mode, as described below.

In the first variation, the magnification factor in the motion mode is less than the magnification factor in the stability mode. Thus, the method of the first preferred embodiment includes automatically decreasing the magnification factor of the medical probe upon significant motion of the medical probe and the subject patient. As used in this document, the phrase magnification factor means the ratio of the size of the physical feature of the subject patient to the size of an image of the physical feature on a display. By decreasing the magnification factor (or “zooming out”), the user can more quickly reorient to the subject patient if the target anatomy is lost from the magnified field of view. Further, if the user intentionally changes probe location, this obviates the need for the user to explicitly adjust the image zoom or scale during the change. The ‘scout’ view may be simply a reduced-magnification image, or it may be a different image mode, e.g. a series of three 2D-images taken on orthogonal planes. The system preferably provides a ‘scout’ view in the motion mode that provides a broader field of view. Likewise, the method of the first preferred embodiment includes automatically increasing the magnification factor of the medical probe, to either the initial value before entering the motion mode or to any other suitable value, upon approximate stability of the medical probe and the subject patient. As used in this document, the terms “significant motion” and “approximate stability” are relative terms and are defined as either side of the threshold explained above.

In a second variation, the frame rate (defined as the measurement of how quickly an imaging device produces unique consecutive images called frames) in the motion mode is greater than the frame rate in the stability mode, and the image resolution (defined as ability to resolve small anatomic features of the subject patient on the display) in the motion mode is less than the image resolution in the stability mode. Thus, the method of the first preferred embodiment includes automatically increasing the frame rate and decreasing the image resolution of the medical probe upon significant motion of the medical probe and the subject patient. By reducing the image resolution, which allows a corresponding increase in frame rate, the distortion, smearing, and time lag effects are significantly reduced, thus improving the user experience. The image resolution, when the medical probe is in the stability mode, may be high-resolution and/or full 3D, resulting in a frame rate below 30 frames per second (“fps”). In the motion mode, image resolution and frame rate are preferably changed to allow a 30 fps or greater update rate. Likewise, the method of the first preferred embodiment includes automatically decreasing the frame rate and increasing the image resolution of the medical probe, to either the initial values before entering the motion mode or to any other suitable values, upon approximate stability of the medical probe and the subject patient.

In a third variation, which is a combination of the first and second variation, the magnification factor in the motion mode is less than the magnification factor in the stability mode, the frame rate in the motion mode is greater than the frame rate in the stability mode, and the image resolution in the motion mode is less than the image resolution in the stability mode. Thus, the method of the first preferred embodiment includes automatically decreasing the magnification factor, increasing the frame rate, and decreasing the image resolution of the medical probe upon significant motion of the medical probe and the subject patient. By combining these three changes, the user can more quickly reorient to the subject patient and can benefit from an improved user experience. Likewise, the method of the first preferred embodiment includes automatically increasing the magnification factor of the medical probe, decreasing the frame rate, and increasing the image resolution of the medical probe, to either the initial values before entering the motion mode or to any other suitable values, upon approximate stability of the medical probe and the subject patient.

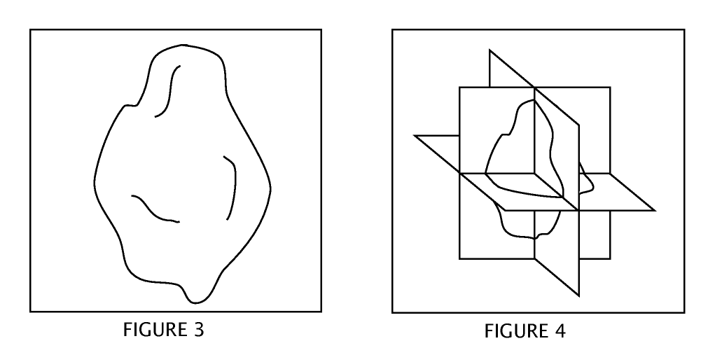

In a fourth variation, the view in the motion mode is different than the view in the stability mode. The different views preferably include a 2D cross-sectional view (as shown in FIG. 3), a 3D segmented view (as shown in FIG. 4), a wire-frame view, and any other suitable view. The motion mode preferably includes the 3D segmented view (or “scout” view), while the stability mode preferably the 2D cross-sectional view. The motion mode and the stability mode may, however, include any suitable mode.

The method of the first preferred embodiment also includes the step of capturing a first series of images of the subject patient with the medical probe in the motion mode (step S18), displaying the first series of images (step S20), capturing a second series of images with the medical probe in the stability mode (step S22), and displaying the second series of images (step S24). The capturing of the images is preferably accomplished with an ultrasonic transducer as described in U.S. patent application Ser. No. 10/840,548 entitled “Ultrasound System including a Handheld Probe” and filed on 06 May 2004 and as described in U.S. patent application Ser. No. 11/229,197 entitled “Integrated Circuit for an Ultrasound System” and filed on 15 Sep. 2005, which are both incorporated by this reference in their entirety. The capturing of the images may, however, be accomplished with any suitable medical device that captures a series of images (2D or 3D) of the subject patient. The displaying of the images may be accomplished by any suitable device, such as a monitor.

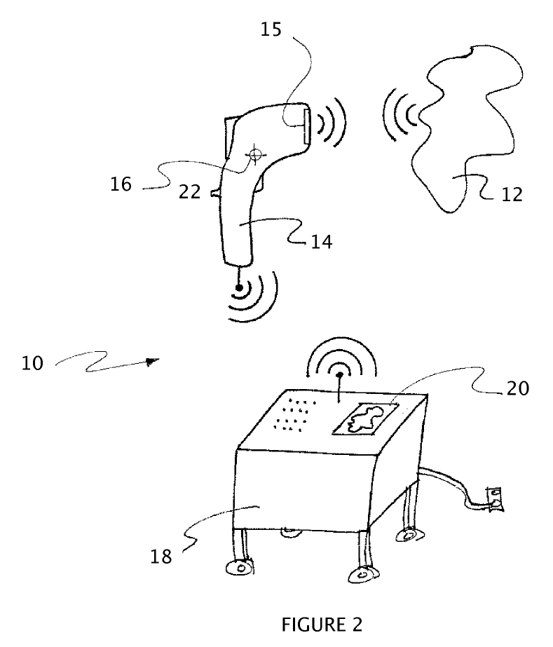

As shown in FIG. 2, a second embodiment includes a system 10 for capturing a series of images of a subject patient 12. The system 10 includes a medical probe 14 and a motion detector 16. The medical probe 14 of the second preferred embodiment functions to capture a series of images of a subject patient 12 with a magnification factor, a frame rate, and an image resolution. The medical probe 14 is preferably an ultrasonic transducer 15, but the medical probe 14 may alternatively be any suitable medical probe to capture a series of images of a subject patient 12.

The motion detector 16 of the second preferred embodiment functions to monitor the motion of the medical probe 14. The motion detector 16 is preferably an accelerometer in the medical probe, but the motion detector 16 may alternatively be a Hall effect sensor in conjunction with magnetic fields, an optical sensor, an RF sensor, an optical sensor, or any other suitable sensor to monitor the relative motion between the subject patient and the medical probe.

The system 10 of the second preferred embodiment also includes a processor 18. The processor 18, which is coupled to the motion detector 16 (through a wired, wireless, or any other suitable connection), functions to determine the motion of the medical probe 14 and to select either a motion mode or a stability mode for the medical probe 14 based on this determination. In one variation, the processor 18 compares the motion to a threshold and selects a motion mode for the medical probe 14 if the motion is greater than the threshold, and selects a stability mode for the medical probe 14 if the motion is less than the threshold. In another variation, the processor 18 allows modification of the threshold by the user. In yet another variation, the processor 18 dynamically modifies the threshold based on the user history, the captured images, the subject patient, or any other suitable factor or combination of factors. The motion mode and the stability mode of the system 10 of the second preferred embodiment are preferably identical to the motion mode and the stability mode of the method of the first preferred embodiment.

The system 10 of the second preferred embodiment also includes a display 20. The display 20 functions to display the series of images capture by the medical probe 14. The display 20 is preferably a monitor, but may be any suitable device or method capable of displaying the series of images captured by the medical probe 14.

The system 10 of the second preferred embodiment may also include a manual trigger 22. The manual trigger functions to allow the user to override the processor 18 and (1) hold the particular mode of the medical probe 14, (2) change the particular mode of the medical probe 14, or (3) any other suitable control of the magnification factor, a frame rate, and an image resolution of the medical probe.

As a person skilled in the art of medical imaging will recognize from the previous detailed description and from the figures and claims, modifications and changes can be made to the preferred embodiments of the invention without departing from the scope of this invention defined in the following claims.

1. A method of capturing a series of images of a subject patient with a medical probe having a magnification factor, a frame rate, and an image resolution, comprising the steps of:
determining a relative motion between a subject patient and a medical probe and comparing the relative motion to a threshold;
if the relative motion is greater than the threshold, selecting a motion mode for the medical probe and capturing a first series of images of the subject patient with the medical probe in the motion mode; and
if the relative motion is less than the threshold, selecting a stability mode for the medical probe and capturing a second series of images of the subject patient with the medical probe in the stability mode.

2. The method of claim 1, wherein the step of determining relative motion or stability between a subject patient and a medical probe includes monitoring the relative motion between the subject patient and the medical probe.

3. The method of claim 2, wherein the step of monitoring includes capturing an initial series of images of the subject patient with the medical probe and analyzing the initial series of images.

4. The method of claim 3, wherein the step of analyzing the initial series of images includes at least one image-processing step selected from the group consisting of tracking motion, correlating frames, and tracking speckles.

5. The method of claim 2, wherein the step of monitoring includes at least one motion-detecting step selected from the group consisting of sensing acceleration forces, sensing Doppler effects, and sensing magnetic fields.

6. The method of claim 1, wherein capturing a first and second series of images of the subject patient include capturing a first and second series of images with an ultrasonic transducer as the medical probe.

7. The method of claim 1, further comprising the steps of displaying the first series of images if the relative motion is greater than the threshold; and displaying the second series of images if the relative motion is less than the threshold.

8. The method of claim 1, wherein the step of selecting a motion mode for the medical probe includes decreasing the magnification factor of the medical probe.

9. The method of claim 1, wherein the step of selecting a motion mode for the medical probe includes increasing the frame rate of the medical probe.

10. The method of claim 9, wherein the step of selecting a motion mode for the medical probe further includes decreasing the image resolution of the medical probe.

11. The method of claim 10, wherein the step of selecting a motion mode for the medical probe further includes decreasing the magnification factor of the medical probe.

12. A medical probe comprising:
an ultrasonic transducer adapted to capture a series of images of a subject patient with a magnification factor, a frame rate, and an image resolution; and
a motion detector adapted to monitor the motion of the medical probe.

13. The medical probe of claim 12, wherein the motion detector is a sensor selected from the group consisting of an accelerometer, a Hall effect sensor, an RF sensor, and an optical sensor.

14. The medical probe of claim 13, further comprising a processor connected to the motion detector, adapted to determine the motion of the medical probe, to compare the motion to a threshold, to select a motion mode for the medical probe if the motion is greater than the threshold, and to select a stability mode for the medical probe if the motion is less than the threshold.

15. The method of claim 14, wherein the processor is adapted to allow modification of the threshold by the user.

16. The method of claim 14, wherein the processor is adapted to dynamically modify the threshold.

17. The method of claim 12, wherein the magnification factor of the medical probe in the motion mode is less than the magnification factor of the medical probe in the stability mode.

18. The method of claim 12, wherein the frame rate of the medical probe in the motion mode is greater than the frame rate of the medical probe in the stability mode.

19. The method of claim 18, wherein the image resolution of the medical probe in the motion mode is less than the image resolution of the medical probe in the stability mode.

20. The method of claim 19, wherein the magnification factor of the medical probe in the motion mode is less than the magnification factor of the medical probe in the stability mode.
